SEQUENCE LISTING (9 sequences)

SEQ ID NO: 1 - DNA, 691 bp, synthetic construct
  source 1..691: mol_type = other DNA; note = FINAL SEQUENCE 1 (variant 1 expression cassette)
taatacgactcactatagggcgaataggagggcgaatgcgaatatgaaaaaatttgtggcgctgctgattaccatgttttttgtggtgagcatggcggcggtggcgcagccggcgagcgcggcgaaatatctgaccggcagctgctgcggcagcacctttagcagcctgagctatggcggcggctgctgccagccgtgctgctgccgcgatccgtgctgctgccgcccggtgacctgccagaccaccgtgtgccgcccggtgacctgcgtgccgcgctgcacccgcccgatttgcgaaccgtgccgccgcccggtgtgctgcgatccgtgcagcctgcaggaaggctgctgccgcccgattacctgctgcccgagcagctgcaccgcggtggtgtgccgcccgtgctgctgggcgaccacctgctgccagccggtgagcgtgcagagcccgtgctgccgcccgccgtgcggccagccgaccccgtgcagcaccacctgccgcaccagcagctgcgcgaaaaaaaaaaaagcgcgccgccgcgcgcatgcgcatgcgcatgcgaacaacaacgcgcagcagcaggcgtgctgctgcgcgtatagctatagcgcggaagaagaagcgccgccgccggcggcgcatcatcatcatcatcattagatagcttttggctcagtcggaagactgggc

SEQ ID NO: 2 - DNA, 1213 bp, synthetic construct
  source 1..1213: mol_type = other DNA; note = FINAL SEQUENCE 3 (variant 3 expression cassette)
taatacgactcactatagggcgaataggagggcgaatgcgaatatgaaaaaatttgtggcgctgctgattaccatgttttttgtggtgagcatggcggcggtggcgcagccggcgagcgcggcgaaatatctgaccggcagctgctgcggcagcacctttagcagcctgagctatggcggcggctgctgccagccgtgctgctgccgcgatccgtgctgctgccgcccggtgacctgccagaccaccgtgtgccgcccggtgacctgcgtgccgcgctgcacccgcccgatttgcgaaccgtgccgccgcccggtgtgctgcgatccgtgcagcctgcaggaaggctgctgccgcccgattacctgctgcccgagcagctgcaccgcggtggtgtgccgcccgtgctgctgggcgaccacctgctgccagccggtgagcgtgcagagcccgtgctgccgcccgccgtgcggccagccgaccccgtgcagcaccacctgccgcaccagcagctgcgcggcgctgaccggcagctgctgcggcagcacctttagcagcctgagctatggcggcggctgctgccagccgtgctgctgccgcgatccgtgctgctgccgcccggtgacctgccagaccaccgtgtgccgcccggtgacctgcgtgccgcgctgcacccgcccgatttgcgaaccgtgccgccgcccggtgtgctgcgatccgtgcagcctgcaggaaggctgctgccgcccgattacctgctgcccgagcagctgcaccgcggtggtgtgccgcccgtgctgctgggcgaccacctgctgccagccggtgagcgtgcagagcccgtgctgccgcccgccgtgcggccagccgaccccgtgcagcaccacctgccgcaccagcagctgcgcggcgaaaaaaaaaaaagcgcgccgccgcgcgcatgcgcatgcgcatgcgaacaacaacgcgcagcagcaggcgtgctgctgcgcgtatagctatagcgcggaagaagaagcgccgccgccggcggcggcggcgaaaaaaaaaaaagcgcgccgccgcgcgcatgcgcatgcgcatgcgaacaacaacgcgcagcagcaggcgtgctgctgcgcgtatagctatagcgcggaagaagaagcgccgccgccggcggcgcatcatcatcatcatcattagatagcttttggctcagtcggaagactgggc

SEQ ID NO: 3 - DNA, 19 bp, synthetic construct
  source 1..19: mol_type = other DNA; note = T7 promoter
taatacgactcactatagg

SEQ ID NO: 4 - DNA, 12 bp, synthetic construct
  source 1..12: mol_type = other DNA; note = 12 nt spacer
gcgaatgcgaat

SEQ ID NO: 5 - DNA, 31 bp, synthetic construct
  source 1..31: mol_type = other DNA; note = TK signal peptide nucleotide sequence (’amino acids)
ggtggcgcagccggcgagcgcggcgaaatat

SEQ ID NO: 6 - DNA, 384 bp, synthetic construct
  source 1..384: mol_type = other DNA; note = Protein KRTAP DNA target sequence
ctgaccggcagctgctgcggcagcacctttagcagcctgagctatggcggcggctgctgccagccgtgctgctgccgcgatccgtgctgctgccgcccggtgacctgccagaccaccgtgtgccgcccggtgacctgcgtgccgcgctgcacccgcccgatttgcgaaccgtgccgccgcccggtgtgctgcgatccgtgcagcctgcaggaaggctgctgccgcccgattacctgctgcccgagcagctgcaccgcggtggtgtgccgcccgtgctgctgggcgaccacctgctgccagccggtgagcgtgcagagcccgtgctgccgcccgccgtgcggccagccgaccccgtgcagcaccacctgccgcaccagcagctgc

SEQ ID NO: 7 - DNA, 123 bp, synthetic construct
  source 1..123: mol_type = other DNA; note = TAIL nucleotide sequence
aaaaaaaaaaaagcgcgccgccgcgcgcatgcgcatgcgcatgcgaacaacaacgcgcagcagcaggcgtgctgctgcgcgtatagctatagcgcggaagaagaagcgccgccgccggcggcg

SEQ ID NO: 8 - DNA, 18 bp, synthetic construct
  source 1..18: mol_type = other DNA; note = HIS tag nucleotide sequence
catcatcatcatcatcat

SEQ ID NO: 9 - DNA, 21 bp, synthetic construct
  source 1..21: mol_type = other DNA; note = Terminator sequence (rrnD)
ggctcagtcggaagactgggc

(19) United States
(12) Patent Application Publication
(10) Pub. No.: US 2026/0151317 A1
(43) Pub. Date: Jun. 4, 2026

(54) PROTEIN CONJUGATE FOR HAIR DYE, A CONSTRUCT, COMPOSITION AND METHOD THEREOF

(71) Applicant: Shefali SABHARANJAK, Bangalore (IN)

(72) Inventor: Shefali SABHARANJAK, Bangalore (IN)

(21) Appl. No.: 18/870,685

(22) PCT Filed: Jun. 6, 2023
(86) PCT No.: PCT/IN2023/050533

§ 371 Date: Dec. 1, 2024

(30) Foreign Application Priority Data
Jun. 7, 2022 (IN) 202241032600

(51) Int. Cl. A61K 8/64; A61K 8/41; A61Q 5/10; C07K 1/107

(52) U.S. Cl. CPC A61K 8/64; A61K 8/41; A61Q 5/10; C07K 1/1077; A61K 2800/4324

(57) ABSTRACT

The present disclosure discloses a Protein KRTAP conjugate linked to at least a tail and optionally a linker; a construct, composition made using said composition; and a method of making and applying the composition. The protein conjugate of the present invention has high modularity and stability especially towards natural dyes and therefore forms an ideal component of hair dye compositions offering carcinogen free formulations unlike currently available hair dye. The present disclosure further discloses a construct formed by the unity of the protein conjugate of the present invention and a dye. The present disclosure further discloses a composition formed of the protein conjugate of the present invention and a method of applying said composition.

### FIELD OF INVENTION

The present disclosure in general relates to a protein conjugate for hair colour. More particularly, the present invention relates to a protein conjugate with improved binding and stability quality towards dye molecules used in hair colour. Further, the present invention also relates to protein constructs, compositions made using the protein conjugate of the present disclosure as well as the method of using the same.

### BACKGROUND OF THE INVENTION

Hair dye happens to be one of the oldest cosmetic technologies. History has it that Egyptians used Hair colour as early as 1500BC. Years later, the Greeks and Romans used plant extracts to colour their hair.

Not much changed until the 1800s, when English chemist William Henry Perkin made an accidental discovery that changed hair dye forever. In an attempt to generate a cure for malaria, Perkins created the first synthesized dye in 1863. The colour was mauve and appropriately named Mauveine. Soon after, his chemistry professor August Hoffman derived a colour-changing molecule from Mauveine (called para-phenylenediamine, or PPD), and it remains the foundation for most permanent hair dyes today.

In 1907, Eugene Schueller created the first chemical dye for commercial purposes. He called it Aureole. It would later be called L'Oréal, as would the company he founded. Whether in the 1800s or in 2022, permanent hair colour is made with harmful chemicals.

Naturally present Hair colour is due to the presence of melanin pigment in the keratinocytes of the hair shaft cortex. Hair colour modification is achieved by removal of cortical melanin pigment through bleaching or addition of artificial pigment through application of a hair dye. Usually, commercial hair dyes are classified into the following three main subtypes according to colour permanence, such as, temporary, semi-permanent, and permanent. Temporary dyes are retained for very short period. The dyes adhere to the hair shaft cuticle via van der Waal force without penetrating into the cortex. Semi-permanent dyes coat the hair shaft cuticle in a similar fashion to temporary dyes while also partially infiltrating the cortex. In contrast, permanent dyes penetrate deeper into the hair shaft, passing through the hair cuticle and are deposited completely into the cortex.

Currently available colours typically contain a system of ammonia (or ethanolamine's in the case of some ammonia-free products), hydrogen peroxide, and p-phenylenediamine. The ammonia pulls apart layers of the hair's proteins, so that the dye can access the hair shaft. Next, hydrogen peroxide bleaches the hair and helps p-phenylenediamine, one of the primary colouring agents, to become trapped in the hair.

These common dye chemicals are associated with negative health effects. Ammonia is a respiratory and asthma irritant, a potential endocrine disruptor, and is persistent in the environment, meaning it sticks around. P-phenylenediamine is associated with birth defects, skin irritation, liver and blood toxicity, and allergic reaction. It is restricted for use in the European Union.

P-phenylenediamine is one of many coal-tar colours, which are derived from petroleum. Coal-tar dyes have been associated with a number of undesirable health effects like eye injury and allergic reactions. Coal tars themselves have been associated with multiple forms of cancer, and some coal-tar dyes have been found to cause cancer in animals. Despite these problems, unlike most colour additives, coal-tar dyes do not need approval from the FDA.

Many dyes can also contain toluene, a well-established neurotoxin, linked to birth defects, pregnancy loss, and allergic reaction. They can also contain resorcinol, a chemical linked to endocrine disruption, meaning it impacts our body's normal hormonal functioning and signaling. Lead acetate, another common ingredient, is linked to neurotoxicity. Conventional dyes can contain many other harmful chemicals like DMDM hydantoin, methylisothiazolinone, and fragrance.

The use of hair dyes is associated with many adverse effects, such as, temporary skin irritation, allergy, skin discoloration, hair breakage, hair color differences. Further, long-term use is also associated with carcinogenicity, mutagenicity and death of hair follicles.

In order to overcome these problems, many hair dye compositions are formulated with natural colouring agents wherein the natural colouring agents are derived from different species of plants.

Usually, keratin filaments comprise the hair shaft in human hair. It is known that KRTAP are family of proteins, which binds to keratins and the cuticle of the hair shaft. Further, these Keratin-associated proteins are grouped into several families. Approximately 100 KRTAP genes have been identified in humans till date with each of the gene classified into a number of families based on their sequence homology and the nature of the repeat structures. KRTAP are useful in the composition of hair dyes.

The Patent Application EP 0404868 B1 discloses a method for dyeing hair with plant dyes. Multiple other attempts have been made by scientists across the world to invent hair colour that is free of harmful chemicals yet none have been successful in offering satisfactory colour intensity, opacity and importantly none can offer a wide range of hues. These are the primary requirements when working with hair colour and yet the primary shortcomings of natural/chemical-free hair colour.

The prior references fail to disclose hair colour which is non-toxic, carcinogen-free, yet long lasting (considering intensity and opacity) and available in a wide range of hues. Therefore, there is a need to overcome at least one of the problems mentioned above.

### SUMMARY OF THE INVENTION

Before the present disclosure for a protein conjugate for hair colour is described, it is to be understood that this application is not limited to the particular systems, and methodologies described, as there can be multiple possible embodiments which are not expressly illustrated in the present disclosure. It is also to be understood that the terminology used in the description is for the purpose of describing the particular versions or embodiments only and is not intended to limit the scope of the present application. This summary is not intended to identify essential features of the claimed subject matter nor is it intended for use in determining or limiting the scope of the claimed subject matter.

An aspect of the present invention discloses a novel Protein KRTAP conjugate having a TAIL and which may additionally have a linker.

Another aspect of the present invention discloses a novel Protein KRTAP construct having a TAIL with modularity to bind with multiple dyes and offers strong and stable binding with both the hair as well as the dye offering a longer lasting effect of the dye once applied over the hair.

Yet another aspect of the present invention discloses the addition of a linker to further increase modularity and stability of the protein conjugate.

Yet another aspect of the present invention discloses a novel protein conjugate represented as: [Protein KRTAP−LINKER (optional)−Protein TAIL], wherein the conjugate may bind with various dyes at the TAIL, LINKER or BOTH, offering a wide range of constructs.

Yet another aspect of the present invention discloses a protein conjugate offering a wide range of constructs which may be typically represented as: [Protein KRTAP−LINKER(x)−Protein TAIL]+[natural Dye]n Wherein, x≥0 and n>1.

Yet another aspect of the present invention discloses a protein conjugate having improved modularity and stability towards the hair and dyes, wherein the dyes are preferably selected from a wide range of natural dyes.

Yet another aspect of the present invention discloses a protein conjugate contained hair colour wherein the protein conjugate is linked to a dye being a pigment, selected from but not limited to a naturally occurring pigment or a chemical pigment

Yet another aspect of the present invention discloses a protein conjugate contained hair construct wherein the protein conjugate is linked to a pigment, selected from but not limited to a naturally occurring pigment or a chemical pigment

Yet another aspect of the present invention discloses a protein conjugate contained composition wherein the protein conjugate is linked to a pigment, selected from but not limited to a naturally occurring pigment or a chemical pigment.

Yet another aspect of the present invention discloses a method of applying hair colour to human hair, wherein the hair colour is formed of the protein conjugate of the present invention.

Yet another aspect of the present invention discloses a protein conjugate expressed using the protein sequence represented as: Promoter->6 nt spacer->ribosome binding site->12 nt spacer->secretion signal sequence->(2 extra amino acids)->(Protein KRTAP)ₘ->(LINKER) x->(TAIL) z->HIS tag->Stop codon->9 nt spacer->Terminator sequence wherein, m>1, x≥0, z>1

Yet another aspect of the present invention discloses a protein conjugate expressed using the protein sequence represented as: Promoter->12 nt spacer->ribosome binding site->secretion signal sequence->2 extra amino acids->(Protein KRTAP)ₘ->(LINKER) x->(TAIL) z->(HIS tag)X->Terminator sequence wherein, m>1, x≥0, z>1

### BRIEF DESCRIPTION OF DRAWINGS

FIG. 1 illustrates the results of experiment conducted as per experimental data according to an embodiment of the present invention;

FIG. 2 illustrates the protein conjugate and construct according to an embodiment of the present invention;

FIG. 3 illustrates the flow chart for the method of spraying the hair dye to the human hair according to an embodiment of the present invention

### DETAILED DESCRIPTION OF THE INVENTION

Some embodiments of the present disclosure, illustrating all its features, will now be discussed in detail. It must also be noted that as used herein and in the appended claims, the singular forms “a”, “an” and “the” include plural references unless the context clearly dictates otherwise. Various modifications to the embodiment will be readily apparent to those skilled in the art and the generic principles herein may be applied to other embodiments. However, one of ordinary skill in the art will readily recognize that the present disclosure for a protein conjugate, construct, composition and method is not intended to be limited to the embodiments illustrated but is to be accorded the widest scope consistent with the principles and features described herein.

Unless otherwise indicated, this disclosure uses the following definitions.

“Nucleotides”/“nt” in the present draft may be represented by adenine (A), cytosine (C), guanine (G), and thymine (T) and may also be Uracil (U)

“Dye” shall mean a natural or synthetic substance used to change the colour of hair and shall include pigments and other colouring agents.

“Conjugate” shall include a “protein conjugate” and shall mean a chemical compound consisting of a sequence of amino acids making up a simple protein to which another non protein group, including a prosthetic group, is attached.

“Linker” shall mean a component that connects or links one unit with another.

“Tail” shall mean a portion that is located at the distal end of the Conjugate.

“Protein Sequence” shall mean the arrangement of amino acids as they occur in a polypeptide chain.

“Polypeptide Chain” Shall mean a polypeptide in a continuous, unbranched chain of amino acids joined by peptide bonds.

“Construct” shall include a “Protein Construct” and shall mean a combination of a Protein Conjugate and a Dye.

“Composition” shall in a non-limiting manner include “Dye, Construct, Conjugate, pH regulators, pigments, fragrant(s), carrier(s), water, industrially acceptable ingredients and additives”.

According to various embodiments that are described below a protein conjugate for hair colour is provided wherein the conjugate is formed of a Protein KRTAP linked to a TAIL and optionally linked to a linker.

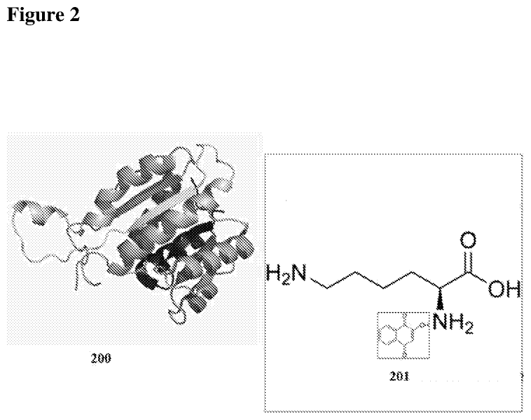

In an embodiment of the present invention a protein conjugate capable of binding with dyes (FIG. 2), said conjugate having structure represented as: [(Protein KRTAP)ₘ−(LINKER)ₓ−(Protein TAIL)_{z}] Where m>1, x≥0, z>1 and wherein the dye is a hair dye (201), being a naturally occurring dye or a chemical dye or a combination of both, preferably the naturally occurring pigment being lawsone, indigo, indican or indigotein, Chlorophyllin, Alizarin red, Purpurin, Xanthin or a combination thereof is disclosed.

In an embodiment of the present invention the protein KRTAP conjugate is expressed using the protein sequence(200):
Promoter->6 nt spacer->ribosome binding site->12 nt spacer->secretion signal sequence->(2 extra amino acids)->(Protein KRTAP)ₘ->(LINKER) x->(TAIL) z->HIS tag->Stop codon->9 nt spacer->Terminator sequence wherein, m>1, x≥0, z>1

In an embodiment of the present invention the protein KRTAP conjugate is expressed using the protein sequence:
T7-promoter->12 nt spacer->SD sequence->TK1887 signal sequence->2 extra amino acids->(Target sequence)X->(HIS tag)X->Terminator sequence.

In an embodiment of the present invention the protein KRTAP conjugate is expressed using the protein sequences as per various embodiments of the present invention wherein:
a. Promoter is T7 promoter: 5′ TAATACGACTCACTATAGG 3′
b. 6 nt spacer being any 6 nucleotides selected for the expression of any of the 22 available amino acids and preferably: GCGAAT
c. ribosome binding site is SD sequence: 5′ AGGAGG
d. 12 nt spacer being any 12 nucleotides selected for the expression of any of the 22 available amino acids and preferably: 5′ GCGAATGCGAAT
e. secretion signal sequence is TK signal peptide sequence (+2 amino acids):

5′ATGAAAAAATTTGTGGCGCTGCTGATTACCATGTTTTTTGTGG
TGAGCATGGCGGC GGTGGCGCAGCCGGCGAGCGCGGCGAAATAT
OR

T or A TAT signal peptide+2 amino acids:

5′ATGAACAACAACGATCTGTTTCAGGCGAGCCGCCGCCGCTTTCTGGC
GCAGCTGGGCGGCCTGACCGTGGCGGGCATGCTGGGCCCGAGCCTGCT
GACCCCGCGCCGCGCGACCGCGGCGCAGGCGGCGACCGATGCGGGCG
GC.
OR

OR
TK signal peptide sequence (+2 amino acids) is represented as below: hGhSecA Signal peptide+2 amino acids:

5′ATGAAAAAAAAAAACATTTATACCATTCGCAAACGCTATGTGG
GCATTGCGACCGTGACCCTGGCGAGCCTGCTGCTGACCGCGACCG
CGAGCCCGGCGGCGATGGCGGGCGGC

f. Protein KRTAP—DNA sequence of the Protein (Target sequence):

5′CTGACCGGCAGCTGCTGCGGCAGCACCTTTAGCAGCCTGAGCT
ATGGCGGCGGCTGCTGCCAGCCGTGCTGCTGCCGCGATCCGTGCT
GCTGCCGCCCGGTGACCTGCCAGACCACCGTGTGCCGCCCGGTGA
CCTGCGTGCCGCGCTGCACCCGCCCGATTTGCGAACCGTGCCGCC
GCCCGGTGTGCTGCGATCCGTGCAGCCTGCAGGAAGGCTGCTGCC
GCCCGATTACCTGCTGCCCGAGCAGCTGCACCGCGGTGGTGTGCC
GCCCGTGCTGCTGGGCGACCACCTGCTGCCAGCCGGTGAGCGTGC
AGAGCCCGTGCTGCCGCCCGCCGTGCGGCCAGCCGACCCCGTGCA
GCACCACCTGCCGCACCAGCAGCTGC

g. LINKER is triple amino acid (amino acid x 3) being expressed by nucleotides selected for the expression of any of the 22 available amino acids
h. protein TAIL is represented as ‘Y’ being a series of amino acids which are in multiples in the protein conjugate and are expressed by nucleotides preferably but not limited to:

AAAAAAAAAAAAGCGCGCCGCCGCGCGCATGCGCATGCGCATGCGAACAAC
AACGCGCAGCAGCAGGCGTGCTGCTGCGCGTATAGCTATAGCGCGGAAGAA
GAAGCGC CGCCGCCGGCGGCG

i. HIS tag built in at C terminal:5′ CATCATCATCATCATCAT
j. Stop Codon: TAG
k. 9 nt Spacer being any 9 nucleotides selected for the expression of any of the 22 available amino acids and preferably: ATAGCTTTT
l. termination sequence (rrnD): GGCTCAGTCGGAAGACTGGGC

Wherein the gene of interest (protein KRTAP) and Modification of Interest (With HIS tag built in at C terminal) can be inserted in a series multiple times

In an embodiment of the present invention the protein conjugate is compatible to bind with various dyes at the TAIL, LINKER or both, offering a wide range of constructs and said dyes are preferably lawsone, indigo, indican or indigotein, Chlorophyllin, Alizarin red, Purpurin, Xanthin or a combination thereof, or a combination thereof.

Another embodiment of the present invention discloses a protein construct formed by the unity of the protein conjugate as and a dye (FIG. 2), said construct having structure represented as: [(Protein KRTAP)ₘ−(LINKER)ₓ−(Protein TAIL)_{z}]+[Dye]ₙ Wherein, m>1, x≥0, z>1 and n>1

In yet another embodiment of the present invention a protein conjugate contained composition is disclosed wherein the protein construct is linked to a dye, the dye being a naturally occurring pigment or a chemical pigment, preferably the naturally occurring pigment being lawsone, indigo, indican or indigotein, Chlorophyllin, Alizarin red, Purpurin, Xanthin or a combination thereof or a combination thereof.

In an embodiment of the present invention protein KRTAP conjugate (200) is linked to the TAIL and LINKER via a covalent bond.

In an embodiment of the present invention protein KRTAP conjugate (200) is linked to the TAIL wherein the protein TAIL is a series of amino acids which are present in multiples within the protein conjugate and are expressed by nucleotides for the expression of any of the 22 available amino acids and preferably:

AAAAAAAAAAAAGCGCGCCGCCGCGCGCATGCGCATGCGCATGCGAA
CAACAACGCGCAGCAGCAGGCGTGCTGCTGCGCGTATAGCTATAGCG
CGGAAGAAGAAGCGC CGCCGCCGGCGGCG.

In another embodiment of the present invention a hair dye composition formed of the protein conjugate of the present invention is disclosed, the composition additionally comprises ethanolamine, antimicrobial agent, hair modifying agent and such other industrially acceptable components.

In another embodiment of the present invention a method of making the protein conjugate of the present invention is provided.

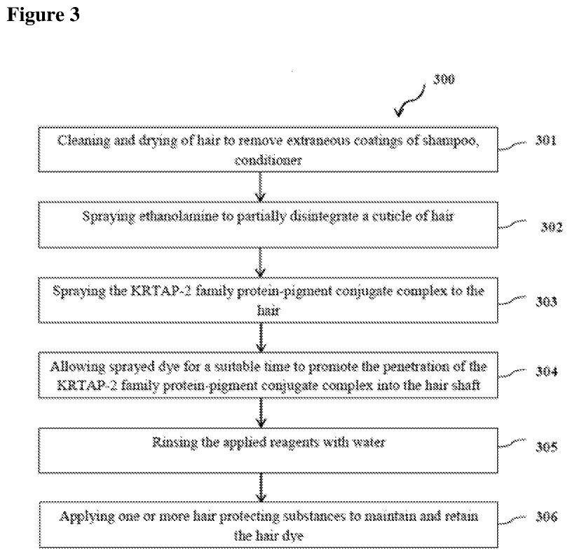

In yet another embodiment a method of applying the hair colour (FIG. 3) formed of said protein conjugate is provided. Wherein, the method (300) comprises the steps:
a. Cleaning and drying the hair to remove the presence of shampoos, conditioners or any other substances (301).
b. Spraying of ethanolamine to mildly disintegrate the cuticle of the hair (302).
c. Spraying the KRTAP-pigment conjugated solution (303).
d. After 10 pumps of spraying, allowing the KRTAP-pigment conjugate within the spray to penetrate into the hair shaft (304).
e. Rinsing off from all the reagents (305).
f. Applying a hair protective substance on the hair to retain the colour, the protective substance is preferably an oil (306).

### Working Examples

Promoter->6 nt spacer->ribosome binding site->12 nt spacer->secretion signal sequence->(2 extra amino acids)->(Target sequence)m->(LINKER)ₓ->(TAIL)_{z}->HIS tag->Stop codon->9 nt spacer->Terminator sequence

### Example 1

In an embodiment of the present invention the protein KRTAP conjugate is expressed using the protein sequence:
Promoter->6 nt spacer->ribosome binding site->12 nt spacer->secretion signal sequence->(2 extra amino acids)->(Protein KRTAP)m->(LINKER) x->(TAIL) z->HIS tag->Stop codon->9 nt spacer->Terminator sequence wherein, m>1, x≥0, z>1
Wherein:
1. Promoter is T7 promoter: 5′ TAATACGACTCACTATAGG 3′
2. 6 nt spacer: GCGAAT
3. ribosome binding site: SD sequence: 5′ AGGAGG
4. 12 nt spacer: 5′ GCGAATGCGAAT
5. secretion signal sequence: TK signal peptide sequence (+2 amino acids):

5′GGTGGCGCAGCCGGCGAGCGCGGCGAAATAT

6. protein KRTAP—DNA sequence of the Protein (Target sequence):

5′CTGACCGGCAGCTGCTGCGGCAGCACCTTTAGCAGCCTGAGCTATGG
CGGCGGCTGCTGCCAGCCGTGCTGCTGCCGCGATCCGTGCTGCTGCCG
CCCGGTGACCTGCCAGACCACCGTGTGCCGCCCGGTGACCTGCGTGCC
GCGCTGCACCCGCCCGATTTGCGAACCGTGCCGCCGCCCGGTGTGCTG
CGATCCGTGCAGCCTGCAGGAAGGCTGCTGCCGCCCGATTACCTGCTG
CCCGAGCAGCTGCACCGCGGTGGTGTGCCGCCCGTGCTGCTGGGCGAC
CACCTGCTGCCAGCCGGTGAGCGTGCAGAGCCCGTGCTGCCGCCCGCC
GTGCGGCCAGCCGACCCCGTGCAGCACCACCTGCCGCACCAGCAGCTG
C

7. LINKER is: GCG
8. TAIL is

5′AAAAAAAAAAAAGCGCGCCGCCGCGCGCATGCGCATGCGCATGCGA
ACAACAACGCGCAGCAGCAGGCGTGCTGCTGCGCGTATAGCTATAGC
GCGGAAGAAGAAGCGCCGCCGCCGGCGGCG

9. HIS tag: CATCATCATCATCATCAT
10. Stop Codon TAG
11. Spacer: ATAGCTTTT
12. Termination sequence (rrnD): GGCTCAGTCGGAAGACTGGGC

FINAL SEQUENCE 1:
5′
TAATACGACTCACTATAGG-GCGAAT-AGGAGG-GCGAATGCGAAT-
ATGAAAAAATTTGTGGCGCTGCTGATTACCATGTTTTTTGTGGTGAGC
ATGGCGGCGGTGGCGCAGCCGGCGAGCGCGGCGAAATAT-
CTGACCGGCAGCTGCTGCGGCAGCACCTTTAGCAGCCTGAGCTATGGC
GGCGGCTGCTGCCAGCCGTGCTGCTGCCGCGATCCGTGCTGCTGCCGC
CCGGTGACCTGCCAGACCACCGTGTGCCGCCCGGTGACCTGCGTGCCG
CGCTGCACCCGCCCGATTTGCGAACCGTGCCGCCGCCCGGTGTGCTGC
GATCCGTGCAGCCTGCAGGAAGGCTGCTGCCGCCCGATTACCTGCTGC
CCGAGCAGCTGCACCGCGGTGGTGTGCCGCCCGTGCTGCTGGGCGACC
ACCTGCTGCCAGCCGGTGAGCGTGCAGAGCCCGTGCTGCCGCCCGCCG
TGCGGCCAGCCGACCCCGTGCAGCACCACCTGCCGCACCAGCAGCTGC
-GCG-
AAAAAAAAAAAAGCGCGCCGCCGCGCGCATGCGCATGCGCATGCGAA
CAACAACGCGCAGCAGCAGGCGTGCTGCTGCGCGTATAGCTATAGCG
CGGAAGAAGAAGCGCCGCCGCCGGCGGCG-CATCATCATCATCATCAT
-TAG-ATAGCTTTT-GGCTCAGTCGGAAGACTGGGC 3′

### Example 2

In another embodiment of the present invention the protein KRTAP conjugate is expressed using the protein sequence as shown in Example 1

With variant 2 having two units of KRTAP+two units of the linker GCG sequence+two units of the tail.

FINAL SEQUENCE 2:
TAATACGACTCACTATAGGGCGAATAGGAGGGCGAATGCGAATATGA
AAAAATTTGTGGCGCTGCTGATTACCATGTTTTTTGTGGTGAGCATGGC
GGCGGTGGCGCAGCCGGCGAGCGCGGCGAAATATCTGACCGGCAGCT
GCTGCGGCAGCACCTTTAGCAGCCTGAGCTATGGCGGCGGCTGCTGCC
AGCCGTGCTGCTGCCGCGATCCGTGCTGCTGCCGCCCGGTGACCTGCC
AGACCACCGTGTGCCGCCCGGTGACCTGCGTGCCGCGCTGCACCCGCC
CGATTTGCGAACCGTGCCGCCGCCCGGTGTGCTGCGATCCGTGCAGCC
TGCAGGAAGGCTGCTGCCGCCCGATTACCTGCTGCCCGAGCAGCTGCA
CCGCGGTGGTGTGCCGCCCGTGCTGCTGGGCGACCACCTGCTGCCAGC
CGGTGAGCGTGCAGAGCCCGTGCTGCCGCCCGCCGTGCGGCCAGCCG
ACCCCGTGCAGCACCACCTGCCGCACCAGCAGCTGC
GCGAAAAAAAAAAAAGCGCGCCGCCGCGCGCATGCGCATGCGCATGC
GAACAACAACGCGCAGCAGCAGGCGTGCTGCTGCGCGTATAGCTATA
GCGCGGAAGAAGAAGCGCCGCCGCCGGCGGCGCATCATCATCATCAT
CATTAGATAGCTTTT GGCTCAGTCGGAAGACTGGGC 3′

### Example 3

In another embodiment of the present invention the protein KRTAP conjugate is expressed using the protein sequence as shown in Example 1

With variant 3 having two units of KRTAP+two units of the linker GCG sequence+two units of the tail.

FINAL SEQUENCE 3:
TAATACGACTCACTATAGGGCGAATAGGAGGGCGAATGCGAATATGA
AAAAATTTGTGGCGCTGCTGATTACCATGTTTTTTGTGGTGAGCATGGC
GGCGGTGGCGCAGCCGGCGAGCGCGGCGAAATATCTGACCGGCAGCT
GCTGCGGCAGCACCTTTAGCAGCCTGAGCTATGGCGGCGGCTGCTGCC
AGCCGTGCTGCTGCCGCGATCCGTGCTGCTGCCGCCCGGTGACCTGCC
AGACCACCGTGTGCCGCCCGGTGACCTGCGTGCCGCGCTGCACCCGCC
CGATTTGCGAACCGTGCCGCCGCCCGGTGTGCTGCGATCCGTGCAGCC
TGCAGGAAGGCTGCTGCCGCCCGATTACCTGCTGCCCGAGCAGCTGCA
CCGCGGTGGTGTGCCGCCCGTGCTGCTGGGCGACCACCTGCTGCCAGC
CGGTGAGCGTGCAGAGCCCGTGCTGCCGCCCGCCGTGCGGCCAGCCG
ACCCCGTGCAGCACCACCTGCCGCACCAGCAGCTGCGCGGCGCTGACC
GGCAGCTGCTGCGGCAGCACCTTTAGCAGCCTGAGCTATGGCGGCGGC
TGCTGCCAGCCGTGCTGCTGCCGCGATCCGTGCTGCTGCCGCCCGGTG
ACCTGCCAGACCACCGTGTGCCGCCCGGTGACCTGCGTGCCGCGCTGC
ACCCGCCCGATTTGCGAACCGTGCCGCCGCCCGGTGTGCTGCGATCCG
TGCAGCCTGCAGGAAGGCTGCTGCCGCCCGATTACCTGCTGCCCGAGC
AGCTGCACCGCGGTGGTGTGCCGCCCGTGCTGCTGGGCGACCACCTGC
TGCCAGCCGGTGAGCGTGCAGAGCCCGTGCTGCCGCCCGCCGTGCGGC
CAGCCGACCCCGTGCAGCACCACCTGCCGCACCAGCAGCTGCGCGGC
GAAAAAAAAAAAAGCGCGCCGCCGCGCGCATGCGCATGCGCATGCGA
ACAACAACGCGCAGCAGCAGGCGTGCTGCTGCGCGTATAGCTATAGC
GCGGAAGAAGAAGCGCCGCCGCCGGCGGCGGCGGCGAAAAAAAAAA
AAGCGCGCCGCCGCGCGCATGCGCATGCGCATGCGAACAACAACGCG
CAGCAGCAGGCGTGCTGCTGCGCGTATAGCTATAGCGCGGAAGAAGA
AGCGCCGCCGCCGGCGGCGCATCATCATCATCATCATTAGATAGCTTT
T GGCTCAGTCGGAAGACTGGGC

### Experimental Data

On January 2023 an experiment was conducted at Protein Design Pvt. Ltd. Located at #517, 4th Cross MSR Nagar Bangalore—560054 to test the stability of protein conjugates as per the present invention.

### Experiment:

The KRTAP+TAIL+His tag protein was expressed in E-coli, purified using standard his-tag purification technologies using a nickel column.

The protein was then dissolved into 1×PBS (phosphate buffered saline) at the concentration of 0.5 mg/ml. Plant extracts—green containing Chlorophyllin and a red extract containing an undefined mix of Alizarin red, Xanthin and Purpurin were dissolve in PBS at 1 mg/ml concentration and finally diluted to 0.1 mg/ml concentration, in 200 μL of the protein solution. The dyes and protein were reacted at 37° Centigrade for 30 mins. The resultant protein mixtures were then run on a 15% SDS polyacrylamide gel to let the free pigments run out.

### Results:

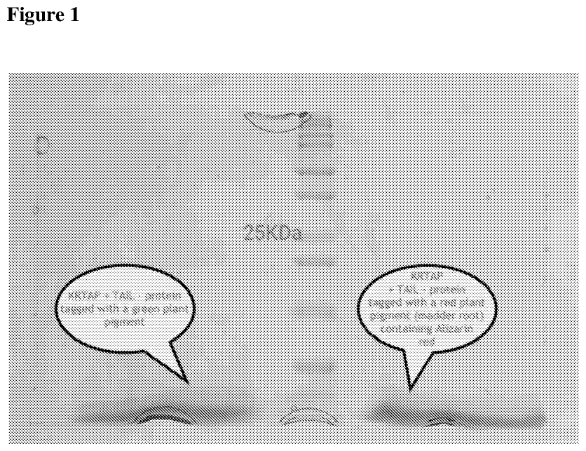

As is seen in FIG. 1, the plant pigments have bound to the protein—seen as a green strip (Chlorophyllin) and red strip (mix of Alizarin red, Xanthin and Purpurin) on the PAGE Gel. The unbound dyes are too small to be retained within the gel and have run out.

### CONCLUSION

The identification of the stable protein conjugates indicates that the protein is capable of binding plant pigments. Similar results can be obtained with other plant pigments, synthetic pigments and other coloured dyes to create safe, protein-based hair dyes.

### Utility

a. The present invention discloses a protein conjugate having improved modularity to bind to versatile dyes especially a large number of natural dyes.
b. The present invention discloses a protein conjugate having improved stability required to bind to natural dyes and to offer a long-lasting effect of the dye whether natural or chemical.
c. The present invention discloses a protein conjugate having flexibility of the number of linkers enabling desired modularity and strength of the protein conjugate.
d. The present invention discloses a hair dye composition formed using the protein conjugate and natural dyes offering a carcinogen and toxin-free composition.

The foregoing descriptions of the invention have been set merely to illustrate the invention and are not intended to be limiting. Since modifications of the disclosed embodiments incorporating the spirit and substance of the invention may occur to person skilled in the art, the invention should be construed to include everything within the scope of the disclosure.

1. A protein conjugate capable of binding with dyes, said conjugate having structure represented as: [(Protein KRTAP)ₘ−(LINKER)ₓ−(Protein TAIL)z] Where m>1, x≥0, z>1 and wherein the dye is a hair dye, being a naturally occurring dye or a chemical dye or a combination of both, preferably the naturally occurring pigment being lawsone, indigo, indican or indigotein, Chlorophyllin, Alizarin red, Purpurin, Xanthin or a combination thereof.

2. A protein construct formed by the unity of the protein conjugate as claimed in claim 1 and a dye, said construct having structure represented as: [(Protein KRTAP)ₘ−(LINKER)ₓ−(Protein TAIL)_{z}]+[Dye]ₙ Wherein, m>1, x≥0, z>1 and n>1.

3. A hair dye composition formed of the protein construct as claimed in claim 2 wherein the protein construct is linked to a dye, being a naturally occurring pigment or a chemical pigment, preferably the naturally occurring pigment being lawsone, indigo, indican or indigotein, Chlorophyllin, Alizarin red, Purpurin, Xanthin or a combination thereof, or a combination thereof.

4. The protein conjugate claimed in claim 1 wherein the conjugate is compatible to bind with various dyes at the TAIL, LINKER or both, offering a wide range of constructs having structure as represented in claim 2 and said dyes are preferably lawsone, indigo, indican or indigotein, or a combination thereof.

5. The hair dye composition as claimed in claim 3, wherein the composition comprises of the protein construct as claimed in claim 2, ethanolamine, antimicrobial agent, hair modifying agent and such other industrially acceptable components and wherein the protein conjugate is linked to a dye, selected from but not limited to a naturally occurring pigment or a chemical pigment.

6. The protein conjugate as claimed in claim 1 is expressed using the protein sequence represented as: Promoter->6 nt spacer->ribosome binding site->12 nt spacer->secretion signal sequence->(2 extra amino acids)->(Protein KRTAP)ₘ->(LINKER) x->(TAIL) z->HIS tag->Stop codon->9 nt spacer->Terminator sequence wherein, m>1, x≥0, z>1

7. The protein conjugate as claimed in claim 1 expressed using the protein sequence represented as: Promoter->12 nt spacer->ribosome binding site->secretion signal sequence->2 extra amino acids->(Protein KRTAP)ₘ->(LINKER) x->(TAIL) z->(HIS tag)->Terminator sequence wherein, m>1, x≥0, z>1

8. The protein conjugate as claimed in claim 1 is expressed using the protein sequence as claimed in claims 6 and 7, wherein:
a. Promoter is T7 promoter: 5′TAATACGACTCACTATAGG 3′
b. 6 nt spacer being any 6 nucleotides selected for the expression of any of the 22 available amino acids and preferably: GCGAAT
c. ribosome binding site is SD sequence: 5′ AGGAGG
d. 12 nt spacer being any 12 nucleotides selected for the expression of any of the 22 available amino acids and preferably: 5′ GCGAATGCGAAT
e. secretion signal sequence is TK signal peptide sequence (+2 amino acids):
5′ATGAAAAAATTTGTGGCGCTGCTGATTACCATGTTTTTTGTGGTGAG
CATGGCGGC GGTGGCGCAGCCGGCGAGCGCGGCGAAATAT
OR
T or A TAT signal peptide+2 amino acids:
5′ATGAACAACAACGATCTGTTTCAGGCGAGCCGCCGCCGCTTTCTGGC
GCAGCTGGGCGGCCTGACCGTGGCGGGCATGCTGGGCCCGAGCCTGCT
GACCCCGCGCCGCGCGACCGCGGCGCAGGCGGCGACCGATGCGGGCG
GC.
OR
TK signal peptide sequence (+2 amino acids) is represented as below:
hGhSecA Signal peptide+2 amino acids:
5′ATGAAAAAAAAAAACATTTATACCATTCGCAAACGCTATGTGGGCA
TTGCGACCGTGACCCTGGCGAGCCTGCTGCTGACCGCGACCGCGAGCC
CGGCGGCGATGGCGGGCGGC
f. Protein KRTAP—DNA sequence of the Protein (Target sequence):
5′CTGACCGGCAGCTGCTGCGGCAGCACCTTTAGCAGCCTGAGCTATGG
CGGCGGCTGCTGCCAGCCGTGCTGCTGCCGCGATCCGTGCTGCTGCCG
CCCGGTGACCTGCCAGACCACCGTGTGCCGCCCGGTGACCTGCGTGCC
GCGCTGCACCCGCCCGATTTGCGAACCGTGCCGCCGCCCGGTGTGCTG
CGATCCGTGCAGCCTGCAGGAAGGCTGCTGCCGCCCGATTACCTGCTG
CCCGAGCAGCTGCACCGCGGTGGTGTGCCGCCCGTGCTGCTGGGCGAC
CACCTGCTGCCAGCCGGTGAGCGTGCAGAGCCCGTGCTGCCGCCCGCC
GTGCGGCCAGCCGACCCCGTGCAGCACCACCTGCCGCACCAGCAGCTG
C
g. LINKER is tripple amino acid (amino acid x 3) being expressed by nucleotides selected for the expression of any of the 22 available amino acids
h. protein TAIL is represented as ‘Y’ being a series of amino acids which are in multiples in the protein conjugate and are expressed by nucleotides preferably but not limited to:
AAAAAAAAAAAAGCGCGCCGCCGCGCGCATGCGCATGCGCATGCGAA
CAACAACGCGCAGCAGCAGGCGTGCTGCTGCGCGTATAGCTATAGCG
CGGAAGAAGAAGCGC CGCCGCCGGCGGCG
i. HIS tag built in at C terminal: 5′ CATCATCATCATCATCAT
j. Stop Codon: TAG
k. 9ntSpacer being any 9 nucleotides selected for the expression of any of the 22 available amino acids and preferably: ATAGCTTTT
l. termination sequence (rrnD): GGCTCAGTCGGAAGACTGGGC

9. The protein conjugate as claimed in claim 1 wherein the protein TAIL is a series of amino acids which are present in multiples within the protein conjugate and are expressed by nucleotides for the expression of any of the 22 available amino acids and preferably:
AAAAAAAAAAAAGCGCGCCGCCGCGCGCATGCGCATGCGCATGCGAA
CAACAACGCGCAGCAGCAGGCGTGCTGCTGCGCGTATAGCTATAGCG
CGGAAGAAGAAGCGC CGCCGCCGGCGGCG.

10. A method of applying the hair colour formed of said protein conjugate is provided. Wherein, the method comprises the steps of:
a. Cleaning and drying the hair to remove the presence of shampoos, conditioners or any other substances.
b. Spraying of ethanolamine to mildly disintegrate the cuticle of the hair.
c. Spraying the KRTAP-pigment conjugated solution.
d. After 10 pumps of spraying, allowing the KRTAP-pigment conjugate within the spray to penetrate the hair shaft.
e. Rinsing off from all the reagents.
f. Applying a hair protective substance on the hair to retain the colour, the protective substance is preferably an oil.
